(19) United States
(12) Patent Application Publication
(10) Pub. No.: US 2017/0367570 A1
(43) Pub. Date: Dec. 28, 2017

(54) FLUID DISTRIBUTOR FOR A REPROCESSING DEVICE FOR SURGICAL INSTRUMENTS

(71) Applicant: OLYMPUS WINTER & IBE GMBH, Hamburg (DE)

(72) Inventor: Ole TOBIAN, HenstedtUlzburg (DE)

(73) Assignee: OLYMPUS WINTER & IBE GMBH, Hamburg (DE)

(21) Appl. No.: 15/681,739

(22) Filed: Aug. 21, 2017

Related U.S. Application Data
(63) Continuation of application No. PCT/EP2016/052622, filed on Feb. 8, 2016.

(30) Foreign Application Priority Data
Feb. 26, 2015 (DE) 102015203429.1

(51) Int. Cl. A61B 1/12

(52) U.S. Cl. CPC A61B 1/123; A61B 1/125

(57) ABSTRACT

A fluid distributor for a reprocessing device for reprocessing surgical instruments. The fluid distributor including: a first fluid routing body having a first fluid hole, the first hole having a first inlet and a first outlet, and a second fluid routing body that has at least two second fluid holes, each of the at least two second fluid holes having a second inlet and a second outlet; wherein the first fluid routing body and the second fluid routing body are rotatable relative to each other about an axis of rotation such that the first fluid hole communicates fluidically in sequence with each of the at least two second fluid holes.

### CROSS-REFERENCE TO RELATED APPLICATION

The present application is a continuation of PCT/EP2016/052622 filed on Feb. 8, 2016, which is based upon and claims the benefit to DE 10 2015 203 429.1 filed on Feb. 26, 2015, the entire contents of each of which are incorporated herein by reference.

### BACKGROUND

### Field

The present application relates to a fluid distributor for a reprocessing device for reprocessing surgical instruments, such as endoscopes. Moreover, the present application relates to a reprocessing device for reprocessing surgical instruments, such as endoscopes, as well as a use of a corresponding fluid distributor in a reprocessing device.

### Prior Art

It is known in the prior art that endoscopes are used to diagnose and treat diseases. For the use of endoscopes as well as other surgical appliances these surgical instruments must be reprocessed, i.e., cleaned and disinfected, after being used on a patient.

In the case of the reprocessing of surgical instruments, legal and clinical regulations must be strictly adhered to so that the components inside, such as endoscope channels and on the surface of the surgical instruments, such as endoscopes, are disinfected and must be free of germs, bacteria, etc.

Reprocessing devices of surgical instruments, such as endoscopes, have apparatuses which clean and disinfect the outer surfaces of the surgical instruments as well as the inner channels and channel systems using appropriate liquids. Washing cycles are normally provided for this. In particular, in the case of the inner channel washing of a surgical instrument, such as of an endoscope or respectively a flexible endoscope, the sanitary result of the reprocessing depends on the flow volume of a reprocessing fluid. Liquids or gases can serve as the reprocessing fluid.

Moreover, a reprocessing apparatus from Olympus Winter & Ibe GmbH, Hamburg, under the name ETD is known for reprocessing and disinfecting flexible endoscopes. In this apparatus, the channels of the endoscopes are washed with a reprocessing fluid or respectively washing, cleaning and/or disinfecting liquids through mechanical cleaning and disinfection of, such as, flexible endoscopes.

In medicine, stringent demands are placed on the reprocessing of surgical instruments in a reprocessing appliance, such as a cleaning and disinfecting appliance, after they have been used. The reprocessing typically comprises the steps of washing, disinfecting as well as drying the endoscopes. One or two washing or pre-washing stages precedes disinfection. This is followed by rinsing processes with clear water and additional drying steps. For washing and disinfecting, one or more chemicals for disinfecting are added to the washing medium, or respectively cleanser. Precise dosing is important in this context.

Particular importance is ascribed to the cleaning and disinfection of the endoscope channels when reprocessing the surgical instruments, such as endoscopes. To reprocess the surgical instruments, the channels of the surgical instruments are connected to a rinsing circuit of the reprocessing device. To reprocess the endoscope channels, the channels are, for example, connected to an adapter of a receiving basket in which the endoscope to be cleaned is arranged so that subsequently, when introducing the receiving basket with the endoscope to be cleaned into the rinsing chamber of the reprocessing device, the channels are connected to the rinsing channels of the rinsing circuit or the adapter with a counterpart of the rinsing circuit.

The channels of the endoscope are coupled to a rinsing circuit provided in the rinsing chamber by plug-in couplings or screwed joints, wherein the channels are connected individually and sequentially to lines of the rinsing circuit. In addition, it is possible to connect the adapter and counterpart to each other.

Stop valves, Y-hose connecting pieces or directional valves which are bypassed and joined in a time-consuming manner and frequently involving much loss, exist for supplying fluids, such as liquids and gases to channels to be cleaned, or respectively in the rinsing circuit. In this context, the fluids flow through channels and can be distributed to a plurality of hoses. It is, however, difficult to provide even distribution as well as even dosing.

### SUMMARY

It is an object to present a fluid distributor as well as a reprocessing device for reprocessing surgical instruments, such as endoscopes, by means of which precise distribution and particularly precise dosing of fluids is enabled in order to efficiently reprocess corresponding surgical instruments with these fluids.

Such object can be achieved with a fluid distributor for a reprocessing device for reprocessing surgical instruments, such as endoscopes, with a first fluid routing body which has a first fluid hole that has a first inlet and a first outlet, and with a second fluid routing body that has at least two second fluid holes which have a second inlet and second outlet in each case, wherein the first fluid routing body and the second fluid routing body are rotatable relative to each other about an axis of rotation such that the first fluid hole communicates fluidically in sequence with the at least two second fluid holes.

Since the first fluid hole fluidically communicates sequentially with the at least two second fluid holes, a fluid can be conveyed at chronological intervals through the first fluid hole to the different second fluid holes. This can occur by precisely dosing the supplied fluid, for example by adjusting a settable fluid flow and adjusting a settable time in which a fluidic communication between the first and respective second fluid hole predominates.

If for example there are two second fluid holes, the fluidic communication can always alternate between the first fluid hole and one of the second fluid holes and an other of the second fluid holes. To provide fluidic communication between the first and second fluid hole, a first outlet of the first fluid hole is arranged, such as directly adjacent, or respectively directly before the respective second inlet of the at least two second fluid holes in the fluid delivery direction. The second inlet of the second fluid hole can be the same size as the first outlet of the first fluid hole. This allows a precise adaptation and loss-free transfer of the fluid from the first fluid hole to the second fluid hole.

The fluid holes can extend from an end face of a fluid routing body to an other end face of the fluid routing body. The fluid holes can be arranged parallel to a longitudinal axis of the respective fluid routing body, or its axis of rotation. The first fluid hole, or the first fluid holes, can be arranged parallel to a longitudinal axis of the first fluid routing body, and the at least two second fluid holes can be arranged parallel to a longitudinal axis of the second fluid routing body.

At least two first fluid holes can be provided in the first fluid routing body. This configuration makes it possible to very easily, efficiently and precisely mix different fluids. For example, two first fluid holes and two second fluid holes can be provided, wherein two different fluids are provided through the two first fluid holes, and consistently alternating fluidic communication is provided from the one first fluid hole alternating between the two second fluid holes, and the other first fluid hole can fluidically communicate alternatingly the other way around with the respective other second fluid hole. In this manner, efficient mixtures of two different fluids which are supplied through the first fluid holes to the second fluid holes can be achieved in the second fluid holes.

A mixture of fluids can be achieved when the first and second fluid routing bodies are rotated relatively quickly in relation to each other so that relatively small portions of the respective fluids are always introduced into the at least two second fluid holes.

In a variation, m multiplied by n second fluid holes can be provided in the second fluid routing body, wherein n first fluid holes are provided in the first fluid routing body, wherein n and m are natural numbers, n is greater than 1, m is greater than or equal to 1 and m can be greater than 1.

This makes it possible for a fluid to pass through several lines or channels for reprocessing surgical instruments, and different fluids, or respectively fluids provided with different components, can be provided mixed or unmixed. For example, two first fluid holes can be provided in a first fluid routing body, and four second fluid holes can be provided in a second fluid routing body. Two first fluid holes and six second fluid holes can also be provided, for example.

The first outlet of the first fluid hole, or the first outlets of the first fluid holes, and the second inlets of the second fluid holes can be at the same distance from the axis of rotation. This ensures that when the first and second fluid routing bodies are rotated relative to each other, an overlap between the first and second fluid hole is reliably enabled. An overlap can be between the respective first outlets and the respective second inlets.

The first fluid holes and second fluid holes can be each equidistant from each other on a circle, the midpoint of which is the axis of rotation.

At least two rows of first outlets of the first fluid holes and at least two rows of second inlets of the second fluid holes can be provided, wherein the distance of each row from the first outlets of the first fluid holes to the axis of rotation corresponds to the distances of a row of second inlets of the second fluid holes to the axis of rotation. This allows significantly more fluid lines, or respectively channels, to be supplied with fluid. The respective fluid holes in the rows can be equidistant. They can be on a circle.

A hollow cylinder can be provided in which the first and second fluid routing bodies are arranged. Alternatively, the first or second fluid routing body can be configured as a hollow cylindrical pot which has the first fluid hole, or the first fluid holes, or the second fluid holes in an end face, wherein the other fluid routing body is arranged in the pot. At least one of the fluid routing bodies, i.e., the first or second fluid routing body, can be configured to be rotatable relative to the hollow cylinder. A collar can be provided on the hollow cylinder to enable attachment of the fluid routing body/bodies along the longitudinal axis.

A flat seal can be arranged between an end face of the first fluid routing body and an end face of the second fluid routing body which permits a relative rotary movement of the first fluid routing body to the second fluid routing body. This enables the first and second fluid holes to be sealed, wherein a relative rotary movement of fluid routing bodies to each other is enabled in the same. The flat seal can, for example, be a seal that has Teflon or consists of Teflon.

A shaft seal can be provided which provides a seal between the hollow cylinder and the first and/or second fluid routing body, or which provides a seal between the first or second routing body designed as a hollow cylindrical pot, and the other fluid routing body. The fluid routing bodies, or at least one fluid routing body, can be radially sealed, i.e., on the circumference, by the shaft seal. The shaft seal can contain Teflon, or consist of Teflon.

A reprocessing device is also provided for reprocessing surgical instruments, such as endoscopes, with a fluid distributor described above. The fluid distributor can be arranged in a housing of the reprocessing device that enables the reprocessing device to be connected to external fluid sources or fluid lines. In this context, the hollow cylinder can be connected securely to the housing, or the cylindrical pot can be connected securely to the housing. The hollow cylinder can also be configured so that the outer contour of the hollow cylinder does not have a circular cross-section but is rather rectangular, although the inner diameter in which the fluid routing body is arranged can have a round cross-section which allows a revolving rotary movement.

A fluid distributor is used in a reprocessing device for reprocessing surgical instruments, such as endoscopes.

A method is also provided for operating a reprocessing device for reprocessing surgical instruments, such as endoscopes, using a fluid distributor, wherein at least one fluid is conducted through at least one first fluid hole of a first fluid routing body to at least two second fluid holes of a second fluid routing body, wherein it is provided that the first fluid hole fluidically communicates sequentially with the at least two second fluid holes so that the fluid is introduced sequentially from the first fluid hole into the at least two seconds fluid holes.

The method can entail transferring fluid from at least two first fluid holes into at least two second fluid holes, wherein the first fluid holes can fluidically communicate sequentially with the at least two second fluid holes in that fluidic communication is provided sequentially. Different fluids can be used to provide fluid mixtures in the second fluid holes. Connections are provided at the second fluid holes which are connected for example to channels of an endoscope in order to conduct the fluids through the channels of the endoscope.

### BRIEF DESCRIPTION OF THE DRAWINGS

Further features will become apparent from the description of embodiments together with the claims and the included drawings. Embodiments can fulfill individual characteristics or a combination of several characteristics.

The embodiments will be described below, without restricting the general idea of the invention, based on such embodiments in reference to the drawings, wherein we expressly refer to the drawings with regard to the disclosure of all details that are not explained in greater detail in the text. In the following:

FIG. 1 illustrates a schematic representation of a reprocessing device,

FIGS. 2a-2d illustrate a schematic plan view of a first fluid routing body with two fluid holes in different rotary positions,

FIGS. 3a-3d illustrate a schematic plan view of a second fluid routing body in which fluids are introduced into corresponding fluid holes corresponding to the rotary positions of the first fluid routing body according to FIG. 2,

FIG. 4 illustrates a schematic sectional representation of a fluid distributor in a first embodiment,

FIG. 5 illustrates a schematic three-dimensional representation of a fluid distributor in a second embodiment.

In the drawings, the same or similar types of elements and/or parts are provided with the same reference numbers so that a re-introduction is omitted.

### DETAILED DESCRIPTION

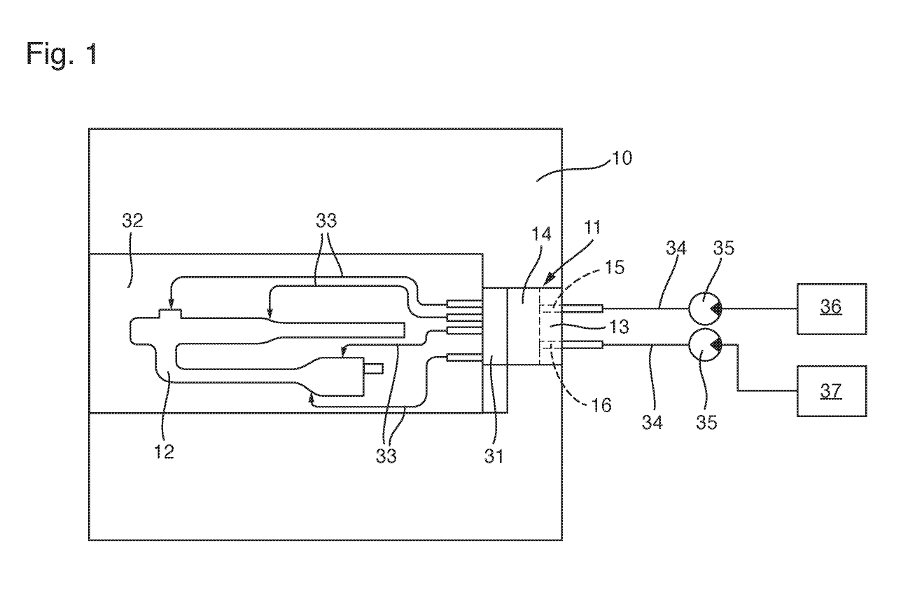

FIG. 1 shows a schematic representation of a reprocessing device 10. In the reprocessing device 10, an endoscope 12 is schematically represented in a basket 32. The endoscope 12 has channels which are to be cleaned and disinfected. Lines 33 are provided for this through which corresponding fluids such as liquids as well as gases can be conducted. In order to conduct corresponding fluids to the endoscope 12, a connection plate 31 is provided on the basket 32 which can be connected to a fluid distributor 11. The lines 33 can be connected without a connection plate 31 directly to the fluid distributor 11. The fluid distributor 11 is supplied a corresponding fluid from pumps 35 via lines 34 from the fluid reservoirs 36 and 37. The fluid distributor 11 has two first fluid holes 15, 16 in a first fluid routing body 13 to which a second fluid routing body 14 is directly connected. Four fluid holes (not shown) are provided in the second fluid routing body and communicate with the connections of the lines 33 to the connection plate 31.

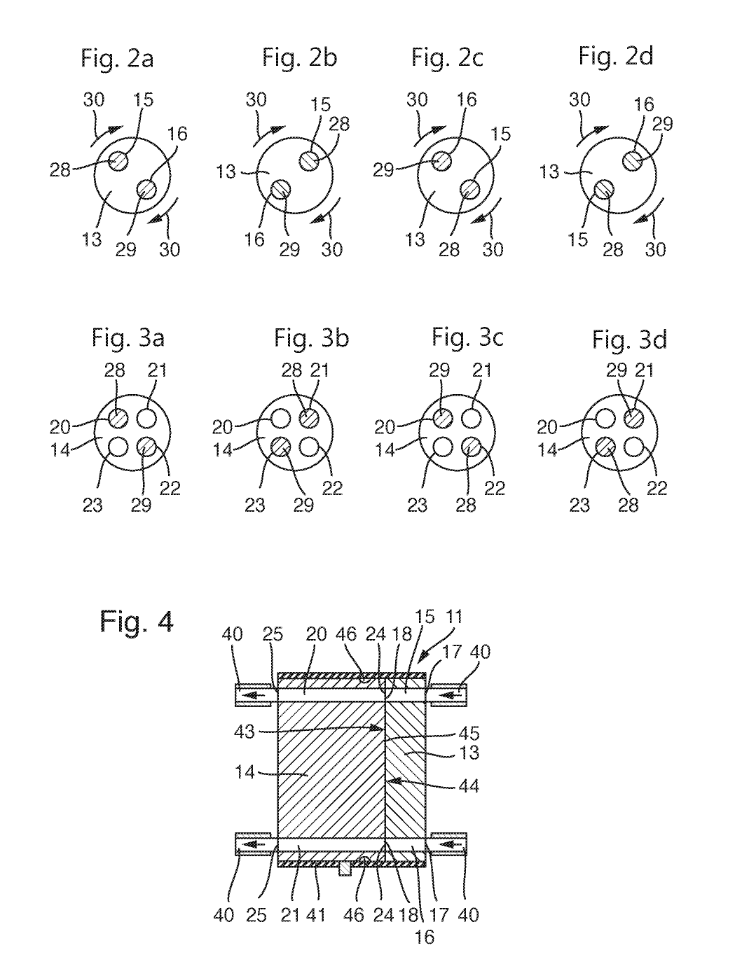

In this exemplary embodiment, the first fluid routing body 13 is rotatable relative to the second fluid routing body 14. Enabling the first routing body 13 to rotate allows the first fluid holes 15 and 16 to alternatingly communicate with the four second fluid holes (not shown) of the second fluid routing body 14. In this exemplary embodiment, two holes always communicate with each other. This is depicted again in FIGS. 2 and 3 in greater detail.

FIGS. 2a-2d show the first fluid routing body 13 in different rotary angles. In a home position which is shown in FIG. 2a, the first fluid routing body 13 is shown which has a first fluid hole 15 and an other first fluid hole 16. Fluids are indicated in each of the fluid holes 15 and 16 and are provided with the reference numbers 28 and 29. The first fluid routing body 13 can be rotated in the direction of the arrow 30, or respectively direction of rotation 30. Starting from the home position in FIG. 2a, a position is reached after a 90° rotation which is shown in FIG. 2b. After another 90° rotation, a position of the first fluid routing body 13 is reached that is shown in FIG. 2c. Correspondingly, the position in FIG. 2d is provided after an additional 90° rotation. To prevent excessive twisting of the supply lines, a reverse rotation can be provided, i.e., opposite the direction of rotation 30, to reach the home position in FIG. 2a.

FIGS. 3a-3d schematically portray the second routing body 14. The second fluid routing body 14 has four second fluid holes 20, 21, 22, 23 which all have the same distance to the midpoint of the cross-section of the second fluid routing body and are equidistant to each other on a circle connecting the midpoints of the second fluid holes 20, 21, 22, 23. Correspondingly, the second fluid holes 20-23 can communicate with the first fluid holes 15, 16 of the first routing body 13 shown in FIGS. 2a-2d depending on the positions after the first fluid routing body 13 is rotated, which are depicted successively in FIGS. 2a, 2b, 2c and 2d.

The holes that communicate with each other can be easily discerned by the fluids indicated in the holes. In the case of FIG. 3a, the fluid 28 is correspondingly let into the hole 20, and the fluid 29 is let into the hole 22. In FIG. 3b, the fluid 28 passes into the second fluid hole 21, and fluid 29 passes into the second fluid hole 23. Correspondingly in FIG. 3c, the fluid 29 passes into the second fluid hole 20, and the fluid 28 passes into the second fluid hole 22. To the extent that the fluid 28 and the fluid 29 are different fluids, a mixture can accordingly be achieved in the respective fluid channels since different fluids are always introduced alternatingly into the second fluid hole 20, 21, 22, 23. With correspondingly short, sequential supplies of fluids, very even dosing and mixing can be achieved.

FIG. 4 schematically shows a sectional view of a fluid distributor 11 according to another embodiment. In this exemplary embodiment, the first fluid routing body 13 and the second fluid routing body 14 are introduced into a hollow cylinder 41. The hollow cylinder 41 can be introduced into a housing of a reprocessing device. FIG. 4 shows that the first fluid hole 15 communicates with the second fluid hole 20, i.e., a fluid can pass in the direction of the arrow from a connection 40 via the first inlet 17 through the first fluid hole 15 and then through the second fluid hole 20 via a second outlet 25 to a connection 40. It can be seen that the first outlet 18 of the first fluid hole 15 lies directly opposite to the second inlet 24 of the second fluid hole 20. The corresponding holds true for the first fluid hole 16 and the second fluid hole 21 which are also shown in FIG. 4.

It can be provided that the first fluid routing body 13 can be rotated about an axis which lies in the plane of the drawing and is horizontal, or the second fluid routing body 14 can correspondingly rotate about this axis of rotation 27 to enable fluidic communication between the first fluid holes 15, 16 and the second fluid holes 20, 21. As long as only two second fluid holes 20, 21 are provided as depicted here in FIG. 4, the respective first fluid hole can accordingly always communicate fluidically with the respective second fluid hole. With the other second fluid holes which are not shown in FIG. 4 since it is a sectional view, a corresponding fluidic communication can be provided as indicated in FIGS. 2 and 3.

To provide a seal, a flat seal 45 for example made of Teflon is provided between the end face 43 of the first fluid routing body 13 and the end face 44 of the second fluid routing body 14. In addition, a radial seal, or respectively a shaft seal 46, can be provided that provides a seal between the second fluid routing body 14 and the hollow cylinder 41 in FIG. 4. A corresponding shaft seal 46 can also be provided in the region of the first fluid routing body 13. This is however not shown in FIG. 4. The shaft seal 46 can be made of Teflon or contain Teflon.

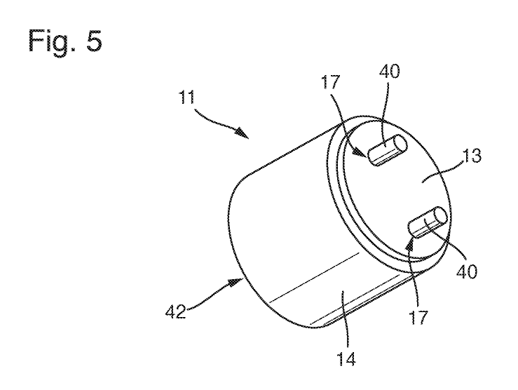

FIG. 5 shows an additional embodiment of a fluid distributor 11. In this exemplary embodiment, the second fluid routing body 14 is designed in the shape of a pot, and the first fluid routing body 13 is introduced into this pot. Since the representation is three-dimensional, the end face 42 is only implied. A corresponding flat seal can also be provided between this end face 42 of the second fluid routing body 14 and an opposing end face of the first fluid routing body 13. The end face 42 which is meant in this context is the one inside the pot.

Here as well, corresponding connections 40 are provided which represent a corresponding first inlet 17 of the first fluid routing body 13, or respectively the first fluid holes 15, 16.

The first and the second fluid routing bodies 13, 14 and the hollow cylinder 41 as well can be made of steel or PEEK, or respectively polyether ether ketone. The cylindrical body can be used as a dosing apparatus. The cylinder in this context is for example designed like a shell or pot and has corresponding throughholes.

The hole midpoints are distributed concentrically around the middle axis.

A rotating insert is located in the pot, or respectively shell, and is connected in a form-fit manner to enable a rotary movement.

The insert can have two or more through-holes which are spaced the same distance from the middle axis of the cylinder as the holes provided in the cylinder itself. This enables the holes to be opened and closed relative to each other during a rotary movement of one of the components. The holes are arranged concentric to each other.

The rotating insert can be driven manually, for example by a locking screw, or automatically by a pneumatic system, or respectively a servomotor, and be moved into a desired position.

Liquid or gaseous media, i.e., fluids, can be conducted by gravitation and/or compressed air, or by pumps through the insert via the cylinders into individual channels and then ultimately into hoses or tubes.

Liquid and gaseous fluids can be dosed thereby. Corresponding holes can be opened or closed by the different rotary positions. The position of the rotations can be controlled or regulated over time, whereby the amount of the flowing fluid can be specifically controlled. Moreover, when there are more than two connections, an efficient mixture of fluids can be enabled.

This enables a very space-saving dosing as well as mixing of the fluids. In addition, cleaning chemicals can be intentionally added.

While there has been shown and described what is considered to be preferred embodiments, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### REFERENCE NUMBER LIST

10 Reprocessing device
11 Fluid distributor
12 Endoscope
13 First fluid routing body
14 Second fluid routing body
15 First fluid hole
16 First fluid hole
17 First inlet
18 First outlet
20 Second fluid hole
21 Second fluid hole
22 Second fluid hole
23 Second fluid hole
24 Second inlet
25 Second outlet
27 Axis of rotation
28 First fluid
29 Second fluid
30 Direction of rotation
31 Connection plate
32 Basket
33 Line
34 Line
35 Pump
36 Fluid reservoir
37 Fluid reservoir
40 Connection
41 Hollow cylinder
42 Face side
43 End face
44 End face
45 Flat seal
46 Shaft seal

What is claimed is:

1. A fluid distributor for a reprocessing device for reprocessing surgical instruments, the fluid distributor comprising:
a first fluid routing body having a first fluid hole, the first hole having a first inlet and a first outlet, and
a second fluid routing body that has at least two second fluid holes, each of the at least two second fluid holes having a second inlet and a second outlet;
wherein the first fluid routing body and the second fluid routing body are rotatable relative to each other about an axis of rotation such that the first fluid hole communicates fluidically in sequence with each of the at least two second fluid holes.

2. The fluid distributor according to claim 1, wherein the first fluid hole comprises at least two first fluid holes.

3. The fluid distributor according to claim 1, wherein the first fluid hole comprises n first fluid holes and the at least two second fluid holes comprises m multiplied by n second fluid holes, wherein n and m are natural numbers, n is greater than 1, and m is greater than or equal to 1.

4. The fluid distributor according to claim 1, wherein the first outlet of the first fluid hole and the second inlets of the at least two second fluid holes are at a same distance from the axis of rotation.

5. The fluid distributor according to claim 3, wherein at least two rows of first outlets of the n first fluid holes and at least two rows of second inlets of the m multiplied by n second fluid holes are provided, wherein a distance of each row from the first outlets to the axis of rotation corresponds to distances of a row of second inlets of the second fluid holes to the axis of rotation.

6. The fluid distributor according to claim 1, further comprising a hollow cylinder in which the first and second fluid routing bodies are arranged.

7. The fluid distributor according to claim 1, wherein one of the first and second fluid routing bodies is configured as a hollow cylindrical pot which has one of the first fluid hole or the at least two second fluid holes in an end face, wherein the other of the first and second fluid routing bodies is arranged in the hollow cylindrical pot.

8. The fluid distributor according to claim 1, further comprising a flat seal arranged between an end face of the first fluid routing body and an opposing end face of the second fluid routing body, the flat seal permitting a relative rotary movement of the first fluid routing body to the second fluid routing body.

9. The fluid distributor according to claim 6, further comprising a shaft seal, the shaft seal providing a seal between the hollow cylinder and the first and/or second fluid routing body.

10. The fluid distributor according to claim 7, further comprising a shaft seal, the shaft seal providing a seal between the first or second fluid routing body configured as the hollow cylindrical pot and the other of the first and second fluid routing body.

11. A reprocessing device for reprocessing surgical instruments, the reprocessing device comprising a fluid distributor according to claim 1.

12. A method of using the fluid distributor according to claim 1 in a reprocessing device for reprocessing surgical instruments, the method comprising:
introducing a first fluid into the first inlet, and
rotating the first fluid routing body relative to the second fluid routing body about an axis of rotation such that the first fluid hole communicates fluidically in sequence with each of the at least two second fluid holes.
